(19) United States
(12) Patent Application Publication
(10) Pub. No.: US 2022/0355084 A1
(43) Pub. Date: Nov. 10, 2022

(54) A GUIDEWIRE MANAGEMENT DEVICE

(71) Applicants: Singapore Health Services PTE LTD, Singapore (SG); Singapore University of Technology and Design, Singapore (SG)

(72) Inventors: Rajkumar Chandran, Singapore (SG); Jerry Kian Teck Lim, Singapore (SG); Seok Hwee Koo, Singapore (SG); Subburaj Karupppasamy, Singapore (SG); Jie Hong Lim, Singapore (SG); Yu Jun Vanessa Poh, Singapore (SG); Pei Jie Janelle Ong, Singapore (SG); Wan Yun LU, Singapore (SG)

(21) Appl. No.: 17/621,642

(22) PCT Filed: Jun. 22, 2020
(86) PCT No.: PCT/SG2020/050347

§ 371 Date: Dec. 21, 2021

(30) Foreign Application Priority Data
Jun. 21, 2019 (SG) 10210905771S

(51) Int. Cl. A61M 25/09; A61M 25/01

(52) U.S. Cl. CPC A61M 25/09041; A61M 25/01; A61M 2025/09125

(57) ABSTRACT

A guidewire management device, a method of manufacturing the guidewire management device and a method for guidewire management are disclosed. The guidewire management device includes a housing through which a lumen of a catheter passes; and a locking mechanism mounted within the housing; wherein the locking mechanism is configured to lock the housing if a guidewire is present in the lumen and to unlock the housing if the guidewire is absent from the lumen. In one embodiment, the locking mechanism comprises a lever structure comprising a first arm connected to a second arm, wherein the first arm is configured to abut the lumen such that it becomes displaceable from a first position to a second position and actuates the second arm to engage a latch member to lock the housing if the guidewire is inserted through the lumen. Conversely, the housing unlocks if the guidewire is removed from the lumen.

### FIELD OF INVENTION

The present invention relates broadly, but not exclusively, to a guidewire management device, a method of manufacturing the guidewire management device and a method for guidewire management.

### BACKGROUND

Central Venous Catheterisation (CVC) is a procedure whereby a catheter is introduced into one of the patient's major veins using the Seldinger Technique in order to administer medications intravenously to the vena cava for critical care. Such a procedure is performed on all Intensive Care Unit (ICU) patients and in critical Accident and Emergency (A&E) cases. CVC is usually done at the jugular vein, subclavian vein, axillary vein or femoral vein.

Complications may arise when using guidewires, for example, the retention of the guidewire. This may lead to arrhythmias, cardiac perforations, thrombosis, infections and even death. If the guidewire is retained, patients would have to go through another vascular surgery to retrieve the guidewire, which may incur additional costs to the patients. Furthermore, retained guidewires are also detrimental to hospitals as they can lead to costly investigations and potential medico-legal litigations.

Current procedural solutions to address the above issue may make use of alterations in the current procedure to prevent the retention of guidewire. These solutions include, but are not limited to, are: asking the clinicians to be more careful; having two people performing the procedure as a means of double checking; introducing a checklist to account for all equipment after the procedure; ensuring the physician grips the proximal end of the guidewire at all times; ensuring the guidewire is not inserted into the body for more than 18 cm; having an active senior physician supervise the procedure and using ultrasound imaging to guide advancement of guidewire.

Despite these procedural solutions, the incidence of retained guidewires continue to rise due to the following reasons: insufficient training and education; lack of confidence; poor technique; lack of awareness of CVC insertion complications among the residents; insufficient supervision and lack of reminders. In other words, procedural solutions are largely dependent on human factors, which may introduce many uncertainties to guarantee success.

On the other hand, current solutions include introducing new technological products to prevent the retention of guidewire. However, such products introduce additional procedures, inaccessibility and inconvenience and still rely on the user to achieve the desired result.

Accordingly, a need exists to provide a guidewire management device that seeks to address some of the above problems.

### SUMMARY

According to a first aspect of the present invention, there is provided a guidewire management device comprising: a housing through which a lumen of a catheter passes; and a locking mechanism mounted within the housing; wherein the locking mechanism is configured to lock the housing if a guidewire is present in the lumen and to unlock the housing if the guidewire is absent from the lumen.

In an embodiment, the housing is configured to enclose suture wings of the catheter when the housing is locked.

In an embodiment, the locking mechanism comprises a lever structure comprising a first arm connected to a second arm, wherein the first arm is configured to abut the lumen and the second arm is configured to be actuated by the first arm.

In an embodiment, the first arm is displaceable from a first position to a second position if the guidewire is inserted through the lumen, the first arm actuating the second arm to engage with a latch member to lock the housing.

In an embodiment, the first arm is displaceable from the second position to the first position if the guidewire is removed from the lumen, the first arm actuating the second arm to disengage from the latch member to unlock the housing.

In an embodiment, the device further comprises a guidewire holder connectable to an end of the lumen, wherein the guidewire holder comprises an elongated tube configured to receive an end of the guidewire.

In an embodiment, the guidewire holder comprises a body comprising a curved track, and wherein the elongated tube is disposed along the curved track.

In an embodiment, the body further comprises a depression configured to receive a thumb of a user, wherein at least a portion of the curved track is disposed in the depression.

According to a second aspect of the present invention, there is provided a method of manufacturing a guidewire management device, the method comprising: forming a housing having a channel for receiving a lumen of a catheter; and mounting a locking mechanism within the housing such that the locking mechanism locks the housing if a guidewire is present in the lumen and unlocks the housing if the guidewire is absent from the lumen.

In an embodiment, forming the housing further comprises forming an enclosure for enclosing suture wings of the catheter when the housing is locked.

In an embodiment, the locking mechanism comprises a lever structure comprising a first arm connected to a second arm, wherein the second arm is configured to be actuated by the first arm, and wherein mounting the locking mechanism comprises disposing the first arm to abut the lumen.

In an embodiment, the method further comprises connecting a guidewire holder to an end of the lumen, wherein the guidewire holder comprises an elongated tube configured to receive an end of the guidewire.

In an embodiment, the method further comprises forming a body of the guidewire holder, the body having a curved track, and disposing the elongated tube along the curved track.

In an embodiment, the method of forming the body further comprises forming a depression for receiving a thumb of a user, wherein disposing at least a portion of the curved track in the depression.

According to a third aspect of the present invention, there is provided a guidewire management method comprising: providing the device as claimed in the first aspect; inserting a guidewire through a lumen of a catheter, the lumen passing through the housing of the device, until the guidewire reaches a predetermined depth; advancing the catheter relative to the guidewire while holding the end of the guidewire using the holder; and after the catheter reaches a predetermined position, withdrawing the guidewire from the lumen using the holder.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1 shows a perspective view of a guidewire management device, according to an example embodiment.

FIG. 2A shows a perspective view of the housing of FIG. 1 in an opened and unlocked state, according to an example embodiment.

FIG. 2B shows a perspective view of the housing of FIG. 2A with its components disassembled.

FIG. 3 shows a side view of a lever structure, according to an example embodiment.

FIGS. 4A and 4B show a cross-sectional view of the locking mechanism in an unlocked and locked position respectively, according to an example embodiment.

FIGS. 4C and 4D show a side view to calculate the angle of displacement of the lever structure, according to an example embodiment.

FIGS. 5A and 5B show a plan view of the guidewire holder, according to an example embodiment.

FIGS. 5C and 5D show a side view to calculate the curvature radius of the curved track, according to an example embodiment.

FIG. 6 shows a flowchart illustrating a method for manufacturing a guidewire management device according to an example embodiment.

FIG. 7 shows a flowchart illustrating a guidewire management method according to an example embodiment.

FIG. 8 shows a flowchart illustrating a method for Central Venous Catheterisation according to an example embodiment.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description. Herein, a guidewire management device is presented in accordance with present embodiments which may address the problem of guidewire retention in Central Venous Catheterization (CVC) through a solution that comprises of a reminder, prevention and fail-safe rolled up into the same solution. The device may be able to inhibit procession of steps beyond the failure of the guidewire safety check, as well as acting as an effective reminder to conduct the guidewire safety check.

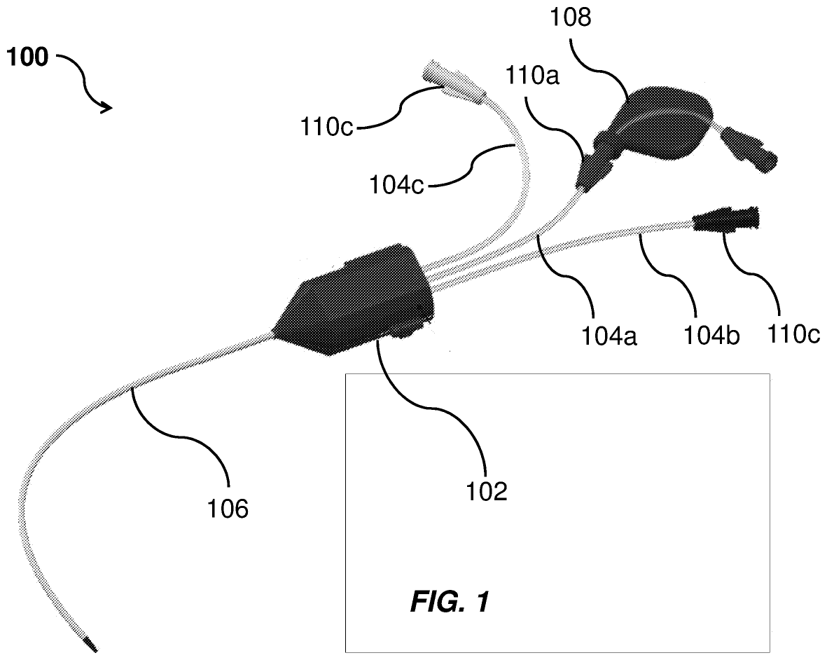

FIG. 1 shows a perspective view of a guidewire management device 100, according to an example embodiment. The device 100 includes a housing 102 through which a lumen 104a of a catheter 106 passes and a guidewire holder 108. It can be appreciated that there may be varying number of lumens with varying dimensions in a catheter due to the different existing catheter manufacturers. In the Figure, the catheter 106 has three lumens 104a, 104b, 104c and the housing 102 may be able to hold the lumens 104a, 104b, 104c. Each of the lumens 104a, 104b, 104c is connected to a respective lumen port 110a, 110b, 110c. The guidewire holder 108 is connected to an end of the lumen 104a via the lumen port 110a.

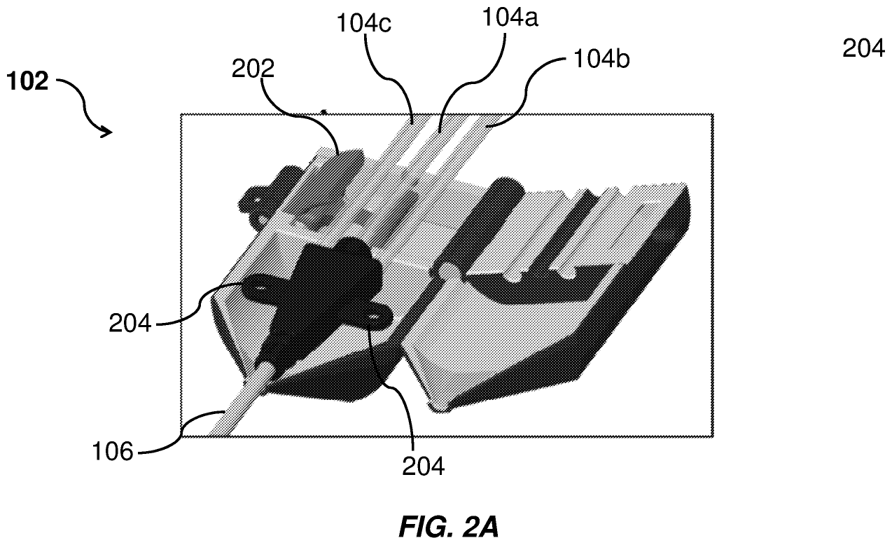

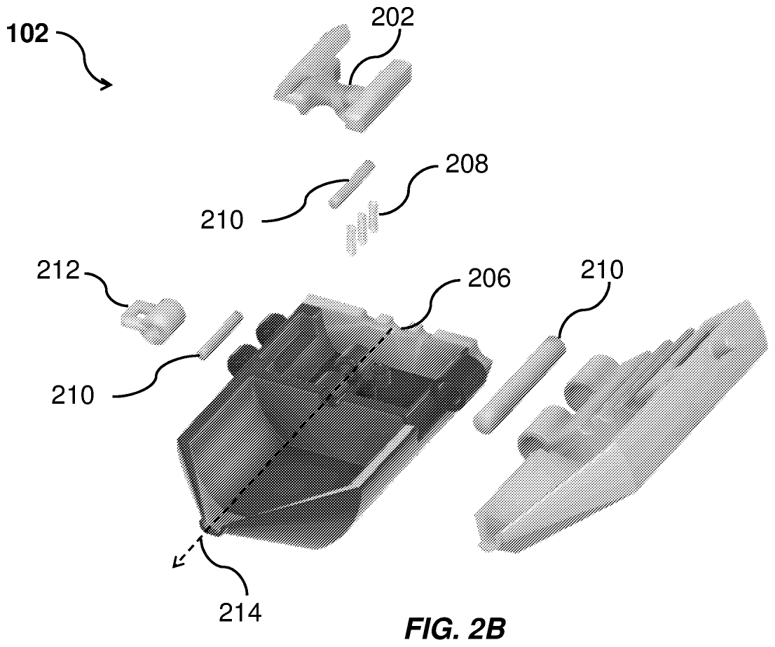

FIG. 2A shows a perspective view of the housing 102 of FIG. 1 in an opened and unlocked state, according to an example embodiment. The housing 102 includes a locking mechanism 202 that is mounted within the housing 102. The locking mechanism 202 is configured to lock the housing 102 if a guidewire is inserted through the lumen 104a and to unlock the housing 102 if the guidewire is removed from the lumen 104a.

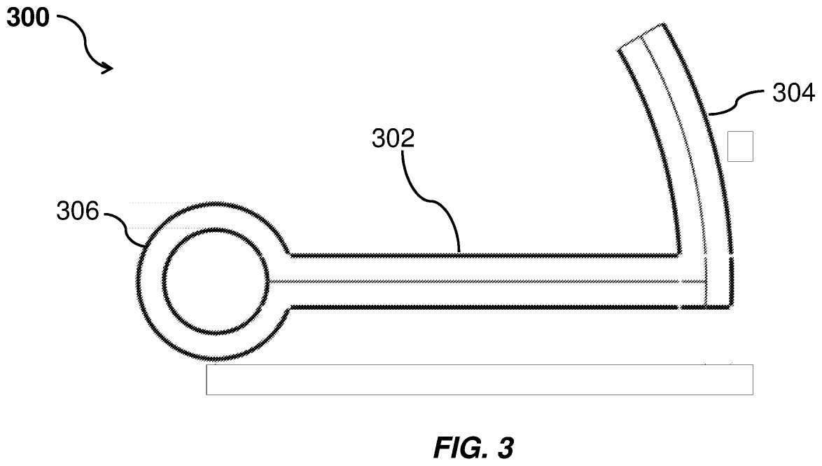

More specifically, the housing 102 detects the presence of a guidewire within the lumen 104a. The housing 102 is affixed to the lumen 104a of the catheter 106, with a lever structure (shown in FIG. 3) pressing against the lumen 104a, depressing it to create an internal width narrower than that of the guidewire. In advancing the guidewire through this depressed portion of the lumen 104a, there is a resultant displacement impacted upon the external width of the lumen 104a. This displacement is translated along the lever structure to create a locking motion within the catheter 106, preventing removal of the housing 102. The lever structure will be explained in detail in FIG. 3.

The housing 102 can be unlocked by reversing the process through the exit of the guidewire from the affected stretch of the lumen 104a. The loss in cross-sectional width can be translated along the lever structure to unlatch a locking pin (not shown), allowing removal of the housing 102. The housing 102 is also designed to include extensions that enclose the suture wings 204 of the catheter 106 when the housing 102 is locked. This may prevent procession of the suturing process and may function as a strong reminder for a user to check if they have removed the guidewire before removing the housing 102 in order to proceed with suturing the catheter 106 to a patient. In other words, the housing 102 may prevent the user from continuing with the surgical procedure until the guidewire is removed.

FIG. 2B shows a perspective view of the housing 102 of FIG. 2A with its components disassembled. In the Figure, the housing 102 further includes a plurality of springs 208 that may allow the lever structure to press against the lumen 104a as described earlier. The housing 102 also includes dowel pins 210 and a latch 212 to open the housing 102 along the side of the catheter for ease of operation. The guidewire is passed through the housing 102 in the direction 214 indicated in the Figure. A lumen channel 206 of the housing 102 may be equal or larger than the lumen's outer diameter. This may prevent unnecessary restriction of the lumen 104a that can affect the flow rate of a priming solution and avoid causing deformation of the lumen channel 206. The lumen 104a may be depressed to a maximum cross-sectional height of 0.8 mm for a detectable change in diameter to occur when the guidewire is advanced through the depressed region.

Detection of the guidewire is based upon a deformation of the lumen 104a and its rebound when the guidewire passes through the lumen channel 206. More specifically, in a neutral position where the guidewire is absent, the plurality of springs 208 pushes the proximal side of the lever structure upwards towards the centre of the housing 102, thereby exerting a force on the lumen 104a, slightly constricting the internal walls of the lumen 104a.

In a non-limiting example, based upon initial measurements done with vernier calipers, the minimum cross-sectional width (with the guidewire present) is approximately 1.3 mm. When the lumen's outer cross-sectional width is constricted to approximately 1.15 mm, passing the guidewire through the lumen 104a may increase the lumen's outer width to approximately 1.43 mm. When a greater force is exerted on the lumen 104a to give an outer cross-sectional width to approximately 0.97 mm, passing the guidewire through the lumen 104a may increase the lumen's outer width to approximately 1.34 mm. Depressing the lumen's outer width to be greater than 1.9 mm may not produce any change in cross-sectional width of the lumen 104a when the guidewire is advanced through it.

Therefore, the range of displacement for detection with the lever structure ranges from approximately 0 mm to 0.37 mm. An ideal restriction of the lumen cross-sectional width would be approximately 1.15 mm, resulting the displacement value of approximately 0.3 mm. By the nature of the lever structure, the spring forces on the end of the lever structure have to balance the resistive force of the lumen 104a.

The spring force required to depress the catheter to the cross-sectional width of 1.15 mm is further determined. A range of springs that having the following dimensions are used, e.g. free length is less than 6 mm, solid length is less than 3 mm and outer diameter is less than 2 mm. The following springs are used: SSC-007A2: Spring Rate=0.716, SSC-008A-2: Spring Rate=1.333, SSC-006A-2: Spring Rate=0.420, SSC-020020-3: Spring Rate=0.333 and SSC-020025-2: Spring Rate=1.349. It was further determined that three springs of type SSC-008A-2, when loaded at a height of 3 mm, were able to constrict the lumen 104a to approximately 1.60 mm. When the guidewire was passed through the lumen 104a, the width of the lumen increased to 1.90 mm, indicating an approximate 0.3 mm change in the cross-sectional width.

A formula to calculate constricted width may be used as below:

Constricted width=(h−h1)mm+1 mm, where h=original height of detection module without introduction of catheter, and h1=height of detection module

After determining the springs used in the system, the amount of spring force acting on the lumen 104a in its different states may be calculated. For example, the force acting upon the lumen 104a in an unlocked state:

Total spring force: F=(1.333)(4.76−2.47)×3=9.16N

This may indicate that the resistive force initiated by the lumen when constricted to a cross-sectional width of 1.60 mm is 9.16 N. Force acting upon lumen in locked state:

Total spring force: F=(1.333)(4.76−2.17)×3=10.36N

This suggests that the total resistive force initiated by the lumen 104a and guidewire when at a combined cross-sectional width of 1.90 mm is approximately 10.36 N.

FIG. 3 shows a side view of a lever structure 300, according to an example embodiment. The lever structure 300 includes a first arm 302 connected to a second arm 304, wherein the first arm 302 is configured to abut the lumen 104a and the second arm 304 is configured to be actuated by the first arm 302. The first arm 302 may include a hollow circular member 306 to abut the lumen 104a.

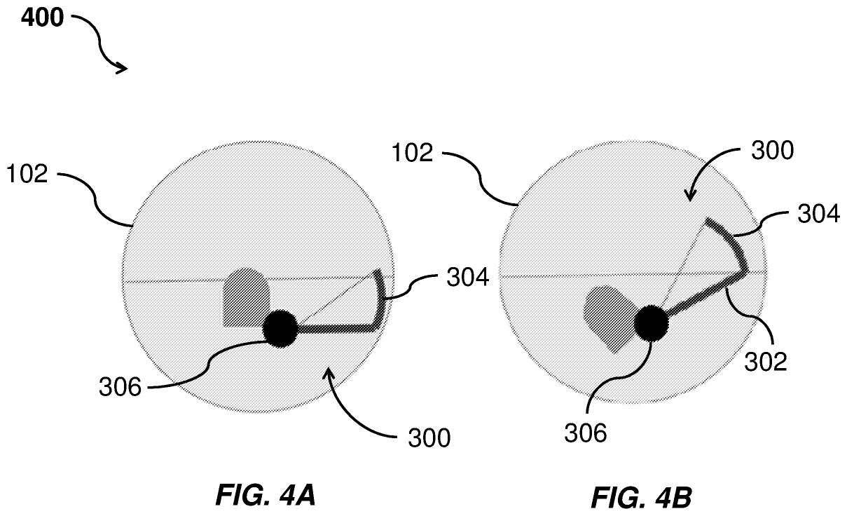

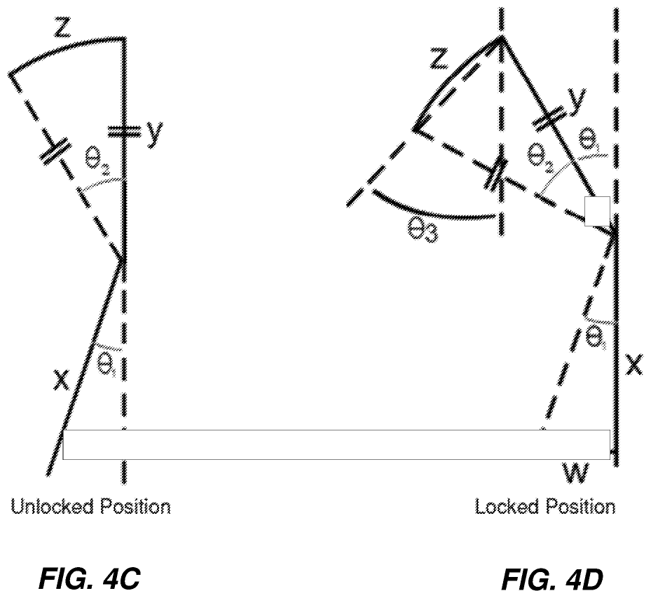

FIGS. 4A and 4B show a cross-sectional view of the locking mechanism in an unlocked and locked position respectively, according to an example embodiment. In FIG. 4B, the first arm 302 may be displaced from a first position to a second position if the guidewire is inserted through the lumen 104a, the first arm 302 actuating the second arm 304 to engage with a latch member to lock the housing 102. In FIG. 4A, the first arm 302 is displaced from the second position to the first position if the guidewire is removed from the lumen 104a, the first arm 302 actuating the second arm 304 to disengage from the latch member to unlock the housing 102. The locking mechanism 202 may be based upon the misalignment of circular projections. The trajectory of the locking mechanism 202 is not concentric to the circular cross-section of the housing 102 as shown in the figures. Such a misalignment may result in locking the housing when the second arm 304 is engaged and latched into the upper circular half of the housing 102. A formula for calculating the angle of displacement of the lever structure is shown below and in FIGS. 4C and 4D.

$$\frac{z}{sin {θ}_{2}} = \frac{y}{sin ( \frac{180 ° - {θ}_{2}}{2} )} \\ z = \frac{sin {θ}_{2}}{sin ( 90 ° - \frac{{θ}_{2}}{2} )} \\ z = y \frac{sin {θ}_{2}}{cos \frac{{θ}_{2}}{2}} \\ {θ}_{3} = ( \frac{180 ° - {θ}_{2}}{2} ) - {θ}_{1} = 90 ° - \frac{{θ}_{2}}{2} - {θ}_{1} \\ {θ}_{1} is dependent on w \\ \frac{w}{x} = sin {θ}_{1} \\ {θ}_{1} = {sin}^{- 1} ( \frac{w}{x} ) \\ where : \\ w is the displacement of lever \\ x is the length of displacement lever \\ y is the length of locking lever \\ z is the length of the hook \\ {θ}_{1} is the angle of displacement \\ {θ}_{2} is the angle of curvature of the hook \\ {θ}_{3} is the angle of hook$$

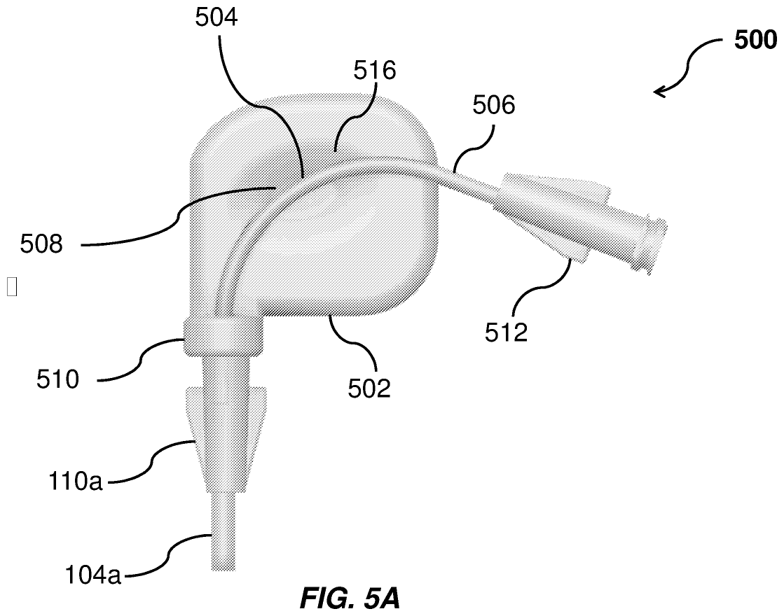

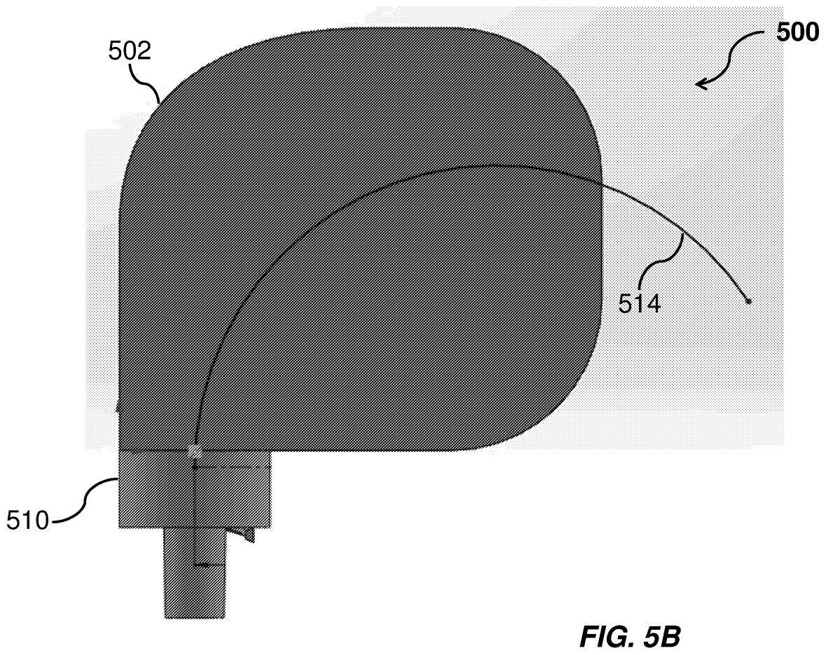

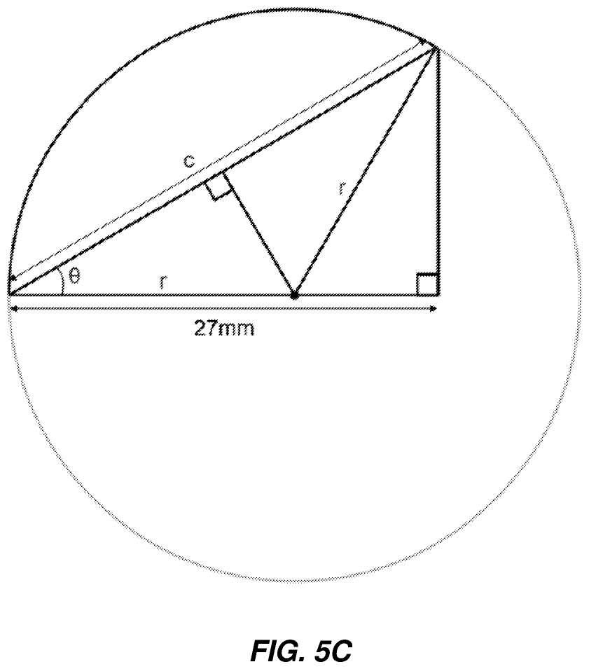

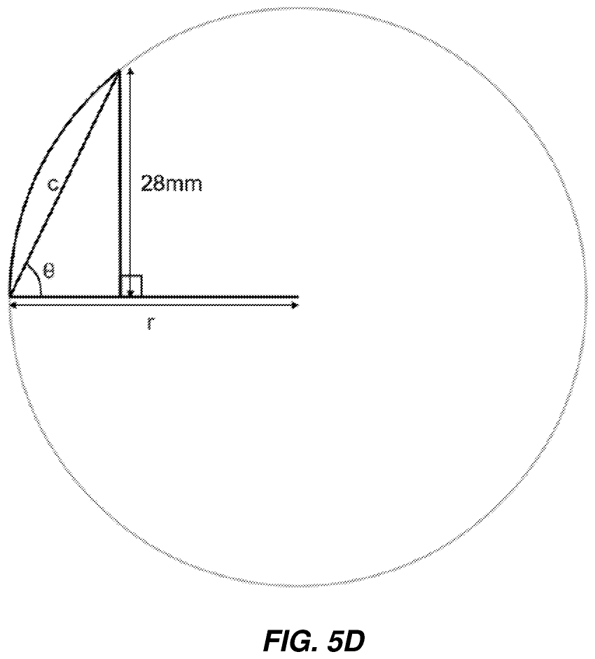

FIGS. 5A and 5B show a plan view of the guidewire holder 500, according to an example embodiment. The guidewire holder 500 includes a body 502 comprising a curved track 504 and an elongated tube 506 configured to receive an end of the guidewire 514. The guidewire holder 500 is connectable to an end of the lumen 104a and the elongated tube 506 is disposed along the curved track 504. The body 502 includes a depression 508 configured to receive a thumb of a user and at least a portion of the curved track 504 is disposed in the depression 508. The guidewire holder 500 also includes a cap 510 configured to receive a lumen port 110a of the catheter 106 and a second lumen port 512 which is connected to the elongated tube 506.

The guidewire holder 500 may prevent the guidewire 514 from slipping back into the vein once it exits the catheter 106. More specifically, the guidewire 514 is curved along the curved track 504 after it exits the lumen 104a. This may create greater resistance and prevent the guidewire 514 from slipping back into the vein. In addition, the guidewire holder 500 may be brightly coloured and have a relatively larger size as compared to the other CVC related instruments. This may provide a visual reminder for the user to remove the guidewire 514. Further, the depression 508 of the guidewire holder 500 is designed such that the user naturally depresses the elongated tube 506 (containing the guidewire 514) when the guidewire holder 500 is removed, thereby pinching onto the guidewire 514 and removing the guidewire 514 together with the guidewire holder 500.

Some dimensions of the guidewire holder 500 may be similar to existing catheter dimension in order to provide simplicity and ensuring effectiveness. This may provide the advantage of being effective and easily interfaced with existing catheters. The dimensions of the elongated tube 506, cap 510 and the second lumen port 512 may be identical to that of the catheter 106. The dimensions and material of the elongated tube 506 may be identical to that of the existing catheter 106 so that the elongated tube 506 can withstand a pre-specified pressure and produce a pre-specified flow rate for priming. The dimensions of the cap 510 may be similar with existing lumen caps since they have to interface with the lumen ports 110a. The second lumen port 512 may be similar with existing lumen ports so that it would be able to interface with the syringes for priming to occur. The guidewire holder 500 may be rotated counterclockwise when being removed. The curved track 504 may curve towards the right so that the user may naturally depress the elongated tube 506 containing the guidewire 514 when removing the guidewire holder 500.

The guidewire holder may include a tab 516 which may be dimensioned such that it is small enough for users to press onto the elongated tube 506 containing the guidewire 514 when removing, but at the same time large enough to allow a sufficient length of guidewire 514 to be threaded through and also to serve as a visual reminder. The outer perimeter of the tab 516 may be determined using a typical size of the top segment of a thumb, which may be approximately 30 mm by 25 mm. Alternatively, the tab 516 outer perimeter may be approximately 32 mm×28 mm (Length×Breadth) due to the incorporation of the depression 508 on the tab 516.

The size of the depression 508 in the middle of the tab 516 was determined by an area of contact between the user's thumb and the tab 516. The size and orientation of the depression 508 may be further improved by having the depression 508 intersect with the curved track 504 with which the guidewire 514 is received.

A circular arc may be used as the shape of the curved track 504 as it does not have any corners and may thus be an ideal path for the guidewire 514 to pass through. As the curved track 504 is curved further, the guidewire 514 may have a higher possibility to stay put as there is greater resistance created by the curvature. However, having a greater resistance may require a greater force to advance the catheter 106 along the guidewire 514 towards the surface of the patient's skin. The angle of curvature of the curved track 504 may be determined such that it may balance between requiring a greater force and having the guidewire 514 to stay put. The angle of curvature may also be the maximum possible radius of an arc that can withstand the desired flow rate and can range between 13.5 mm and 390 mm. Curvature arcs with radiuses greater than 390 mm may cause the guidewire 514 to slip out when the catheter 104 is held vertically while curvature arcs with radiuses lesser than 10 mm may cause kinks in the elongated tube 506 or the guidewire 514 when the catheter 106 is advanced. The angle of curvature may be calculated as follows:

For angles less than

$${tan}^{- 1} ( \frac{28}{27} ) = 46 ° ,$$

the radius can be calculated as shown below and in FIG. 5C

$$cos θ = \frac{c}{2 r} \\ cos θ = \frac{27}{c} \\ Combining both the above equations , \\ \frac{27}{c} = \frac{c}{2 r} \\ 54 r = {c}^{2} \\ c = 3 \sqrt{6 r} \\ Substituting c = 3 \sqrt{6 r} into cos θ = \frac{27}{c} , \\ cos θ = \frac{9}{\sqrt{6 r}} \\ r = \frac{{( \frac{9}{cos θ} )}^{2}}{6}$$

For angles greater than 46°, the radius can be calculated as shown below and in FIG. 5D

$$cos θ = \frac{c}{2 r} \\ sin θ = \frac{28}{c} \\ Combining both the above equations , \\ c = 2 r cos θ = \frac{28}{sin θ} \\ 2 sin θ cos θ = \frac{28}{r} \\ sin 2 θ = \frac{28}{r} \\ r = \frac{28}{sin 2 θ}$$

With the θ values determined earlier, the radiuses were calculated using formulas obtained above. The curvature radius of the curved track 504 may also be determined by using the maximum allowable flow rate through the lumen 104a. For example, for a maximum flow rate of 3100 mLitres/hour, the radius of the curvature is less than 22 mm, or more ideally a radius of approximately 20 mm.

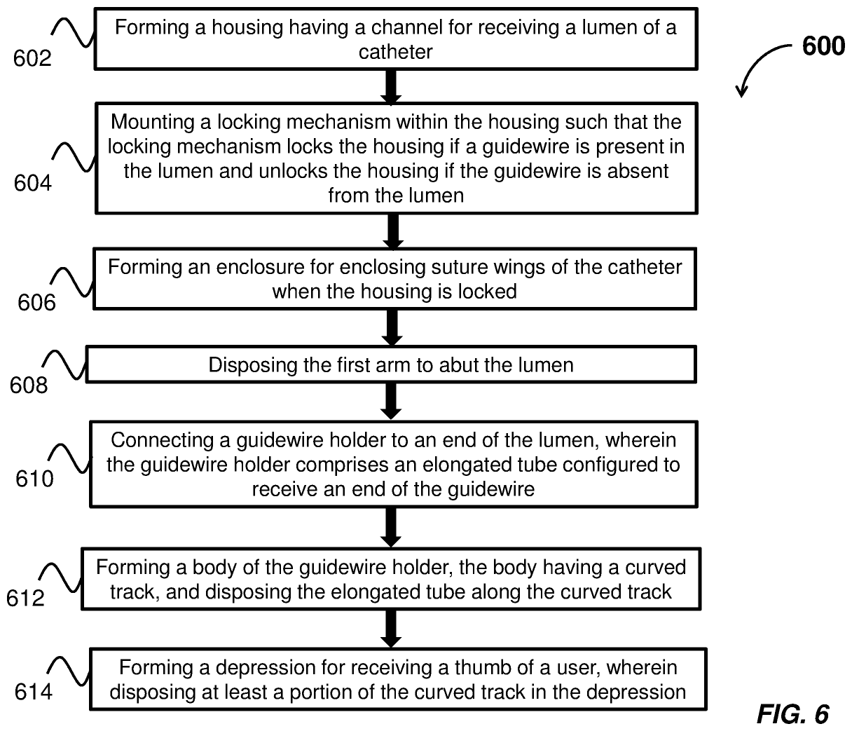

FIG. 6 shows a flowchart 600 illustrating a method for manufacturing a guidewire management device according to an example embodiment. The method comprises at step 602, forming a housing having a channel for receiving a lumen of a catheter. At step 604, the method includes mounting a locking mechanism within the housing such that the locking mechanism locks the housing if a guidewire is present in the lumen and unlocks the housing if the guidewire is absent from the lumen. At step 606, the method for forming the housing includes forming an enclosure for enclosing suture wings of the catheter when the housing is locked. At step 608, the method of mounting the locking mechanism includes disposing the first arm to abut the lumen. At step 610, the method includes connecting a guidewire holder to an end of the lumen, wherein the guidewire holder comprises an elongated tube configured to receive an end of the guidewire. At step 612, the method includes forming a body of the guidewire holder, the body having a curved track, and disposing the elongated tube along the curved track. At step 614, the method of forming the body includes forming a depression for receiving a thumb of a user, wherein disposing at least a portion of the curved track in the depression.

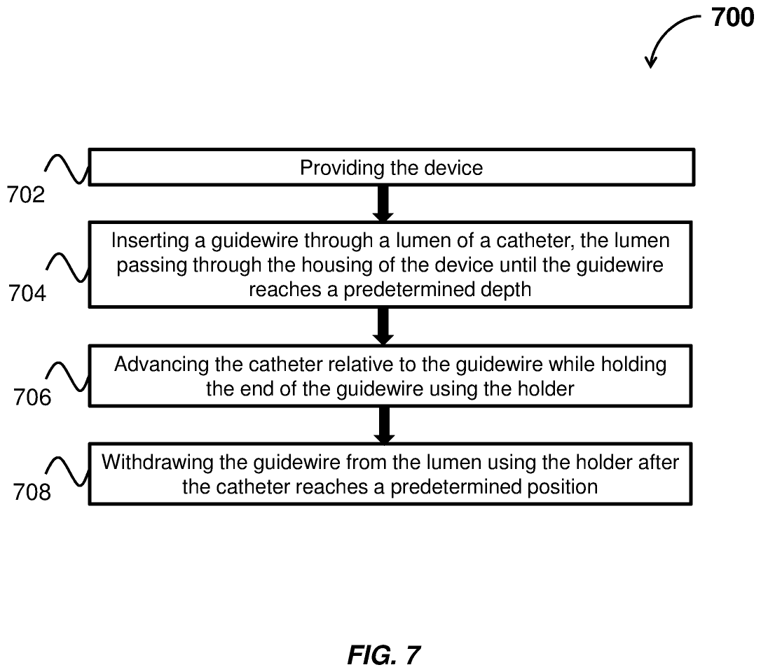

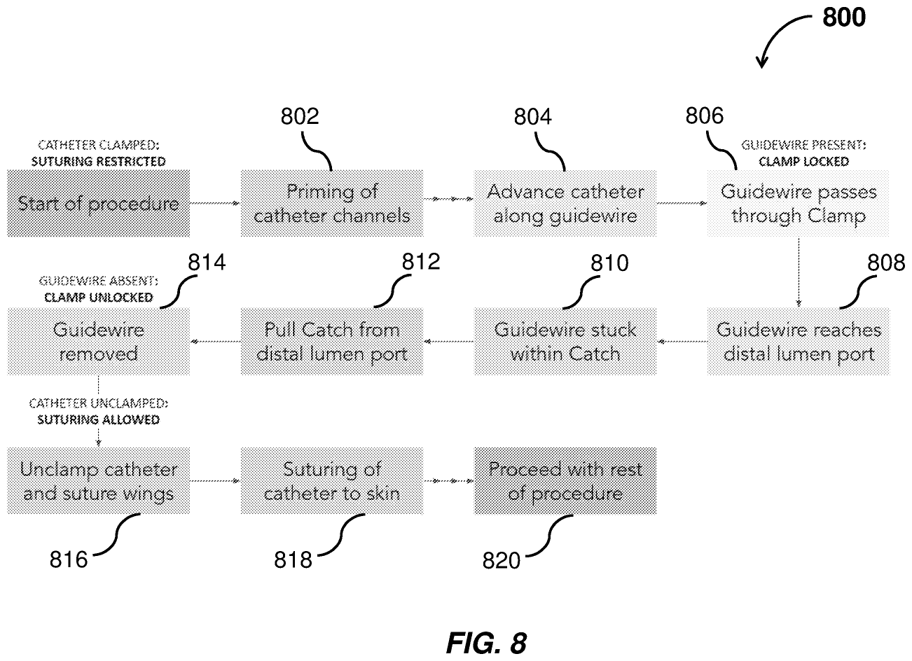

FIG. 7 shows a flowchart 700 illustrating a guidewire management method according to an example embodiment. At step 702, the method includes providing the device as described. At step 704, the method includes inserting a guidewire through a lumen of a catheter, the lumen passing through the housing of the device until the guidewire reaches a predetermined depth. At step 706, the method includes advancing the catheter relative to the guidewire while holding the end of the guidewire using the holder. At step 708, the method includes withdrawing the guidewire from the lumen using the holder after the catheter reaches a predetermined position.

FIG. 8 shows a flowchart 800 illustrating a method for Central Venous Catheterisation (CVC) according to an example embodiment. At step 802, the method includes priming of the catheter channels. At step 804, the method includes advancing the catheter along with the guidewire. At step 806, the method includes passing the guidewire through the clamp (or housing). At this step, the clamp (or housing) is locked. At step 808, the method includes reaching the guidewire to the distal lumen port (or lumen port). At step 810, the method includes engaging the guidewire within the catch (or guidewire holder). At step 812, the method includes pulling the catch (or guidewire holder) from the distal lumen port (or lumen port). At step 814, the method includes removing the guidewire. At this step, the clamp (or housing) is unlocked and suturing is allowed. At step 816, the method includes unclamping the catheter and suture wings. At step 818, the method includes suturing of the catheter to the skin. At step 820, the method includes proceeding with the rest of the CVC procedure.

The guidewire management device as described herein may address the problem of guidewire retention in Central Venous Catheterization (CVC) through a solution that comprises of a reminder, prevention and fail-safe. Current products in the market suggest solutions to the problem of guidewire retention, but do not seamlessly integrate into the existing Seldinger Technique used in CVCs. Each of the elements in the present device targets one major cause of guidewire retention. The housing targets the problem of negligence of the physicians by locking up the suturing wings on the catheter and preventing continuation of the surgical procedure until it detects the removal of the guidewire, and thus may act as a fail-safe mechanism. The guidewire holder targets the problem of the guidewire slipping back into the veins as the catheter is advanced, by securing the guidewire after it exceeds the distal lumen port, which may serve as a visual reminder for physicians to anchor the distal end of the guidewire and allowing for easy removal of the guidewire.

The guidewire holder may also increase impediment to clinicians when carrying out suturing procedure, thus ensuring that the clinicians are reminded of the removal of guidewire, through the need to remove the housing to carry out suturing. This acts as a strong mechanism to enforce compliance to surgical procedure, such that the removal of guidewire is not easily neglected and therefore reducing the risk of the guidewire being retained. The small size and fitted shape of the housing may reduce blockage and enhance user experience. The force impacted upon the lumen in the neutral position (i.e. when the guidewire is absent) may also ensure there is no blockage of fluid flow or guidewire advancement. Accordingly, the force acting on the lumen may also ensure that the fluid flow is blocked when the guidewire is present in the lumen. The dimensions and ergonomics of the guidewire holder may serve as a visual reminder and an aid in removing the guidewire. The curvature of the curved track may provide resistance to the motion of the guidewire and may prevent it from slipping back into the vein once it exits the lumen port, ensuring that it does not hinder the advancement of the catheter into the vein at the same time. Both elements of the guidewire management device are easily integrated into current CVC procedures as the device can be used as attachments at the beginning of the procedure and removed at the end of the procedure without interfering with any other steps during the process, including priming. Adaptation to different catheterization sets and mass production at a low cost may be possible due to the simple design and mechanics of the guidewire management device. While exemplary embodiments have been presented in the foregoing detailed description of the invention, it should be appreciated that a vast number of variations exist.

It should further be appreciated that the exemplary embodiments are only examples, and are not intended to limit the scope, applicability, operation, or configuration of the invention in any way. Rather, the foregoing detailed description will provide those skilled in the art with a convenient road map for implementing an exemplary embodiment of the invention, it being understood that various changes may be made in the function and arrangement of elements and method of operation described in an exemplary embodiment without departing from the scope of the invention as set forth in the appended claims.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

1. A guidewire management device comprising:
a housing through which a lumen of a catheter passes; and
a locking mechanism mounted within the housing;
wherein the locking mechanism is configured to lock the housing if a guidewire is present in the lumen and to unlock the housing if the guidewire is absent from the lumen.

2. The guidewire management device according to claim 1, wherein the housing is configured to enclose suture wings of the catheter when the housing is locked.

3. The guidewire management device according to claim 1 or 2, wherein the locking mechanism comprises a lever structure comprising a first arm connected to a second arm, wherein the first arm is configured to abut the lumen and the second arm is configured to be actuated by the first arm.

4. The guidewire management device according to claim 3, wherein the first arm is displaceable from a first position to a second position if the guidewire is inserted through the lumen, the first arm actuating the second arm to engage with a latch member to lock the housing.

5. The guidewire management device according to claim 4, wherein the first arm is displaceable from the second position to the first position if the guidewire is removed from the lumen, the first arm actuating the second arm to disengage from the latch member to unlock the housing.

6. The guidewire management device according to any one of the preceding claims, further comprising a guidewire holder connectable to an end of the lumen, wherein the guidewire holder comprises an elongated tube configured to receive an end of the guidewire.

7. The guidewire management device according to claim 6, wherein the guidewire holder comprises a body comprising a curved track, and wherein the elongated tube is disposed along the curved track.

8. The guidewire management device according to claim 7, wherein the body further comprise a depression configured to receive a thumb of a user, wherein at least a portion of the curved track is disposed in the depression.

9. A method of manufacturing a guidewire management device, the method comprising:
forming a housing having a channel for receiving a lumen of a catheter; and
mounting a locking mechanism within the housing such that the locking mechanism locks the housing if a guidewire is present in the lumen and unlocks the housing if the guidewire is absent from the lumen.

10. The method according to claim 9, wherein forming the housing further comprises forming an enclosure for enclosing suture wings of the catheter when the housing is locked.

11. The method according to claim 9, wherein the locking mechanism comprises a lever structure comprising a first arm connected to a second arm, wherein the second arm is configured to be actuated by the first arm, and wherein mounting the locking mechanism comprises disposing the first arm to abut the lumen.

12. The method as claimed in according to claim 9, further comprising connecting a guidewire holder to an end of the lumen, wherein the guidewire holder comprises an elongated tube configured to receive an end of the guidewire.

13. The method according to claim 12, further comprising forming a body of the guidewire holder, the body having a curved track, and disposing the elongated tube along the curved track.

14. The method according to claim 13, wherein forming the body further comprises forming a depression for receiving a thumb of a user, wherein disposing at least a portion of the curved track in the depression.

15. A guidewire management method comprising:
providing the device of claim 6;
inserting a guidewire through a lumen of a catheter, the lumen passing through the housing of the device, until the guidewire reaches a predetermined depth;
advancing the catheter relative to the guidewire while holding the end of the guidewire using the holder; and
after the catheter reaches a predetermined position, withdrawing the guidewire from the lumen using the holder.
